(19) United States
(12) Patent Application Publication
(10) Pub. No.: US 2023/0221577 A1
(43) Pub. Date: Jul. 13, 2023

(54) COLOR CHANGING EYEGLASS FRAMES AND ACCESSORIES

(71) Applicant: Awilda Sosa, New York, NY (US)

(72) Inventor: Awilda Sosa, New York, NY (US)

(21) Appl. No.: 18/096,683

(22) Filed: Jan. 13, 2023

Related U.S. Application Data
(60) Provisional application No. 63/297,824, filed on Jan. 10, 2022.

(51) Int. Cl. G02C 5/00; G02C 11/00

(52) U.S. Cl. CPC G02C 5/001; G02C 11/10

(57) ABSTRACT

A dynamic glasses frame is disclosed at least one frame member comprising a color changing element to change from at least one first color to at least one second color in response to a stimulus. The at least one frame element includes a first temple, a second temple, and a frame, and wherein the frame is coupled to one or more lenses. The dynamic elements that allow for the change of colors and/or designs. These can reflect a user's style, individuality, and/or the environment.

### CROSS-REFERENCE TO RELATED APPLICATIONS

The present application claims priority to U.S. Provisional Application No. 63/297,824 filed Jan. 10, 2022, titled “COLOR CHANGING EYEGLASS FRAMES AND ACCESSORIES,” which is hereby incorporated by reference in its entirety.

### TECHNICAL FIELD

Embodiments of the invention relate to systems and methods for color changing eyeglass frames and accessories.

### BACKGROUND

Eyeglasses (referred to herein as glasses and eyeglasses and also including sunglasses and other related eyewear) generally include one or more lenses that a user looks through to correct their vision. These are most often held in place in front of the user's eyes by a frame, which can include one or more arms and can include a connector that is worn over the nose. People with mild, moderate, and severe vision problems often own and wear glasses to correct the issue and allow them to carry out their lives in the same manner as those without vision problems. Even people without vision problems may elect to wear glasses for their aesthetic appeal, on occasion.

Most people that wear glasses own a small number of pairs, which can function for everyday use, backup, reading, computer use, special occasion, sports, and/or sunglasses. While many people would surely appreciate having a pair of glasses for every occasion and need, the majority of people select glasses that have a neutral color for all purpose use and can be used in many different situations. Many people find traditional glasses frames to be boring and static. As such, many individuals would like to have glasses and glasses accessories that are unique, individual, stylish, and dynamic.

Thus, it would be beneficial to have glasses and accessories that are versatile and dynamically reflect a wearer's individuality.

### SUMMARY OF THE INVENTION

This summary is provided to introduce a variety of concepts in a simplified form that is disclosed further in the detailed description of the embodiments. This summary is not intended for determining or limiting the scope of the claimed subject matter.

Glasses frames and accessories disclosed herein can include dynamic elements that allow for them to change colors and/or designs. These can reflect a user's style, individuality, and/or the environment. The dynamic glasses frame may include at least one frame member comprising a color changing element to change from at least one first color to at least one second color in response to a stimulus. The at least one frame element may include a first temple, a second temple, and a frame, wherein the frame is coupled to one or more lenses.

In one aspect, the color changing element is integrated into the at least one frame member.

In one aspect, the color changing element is at least partially embedded into the at least one frame member.

In one aspect, the color changing element is removably coupled with the at least one frame member.

In one aspect, sensing elements may be included with the dynamic glasses frame. The sensing elements may receive the stimulus to change the color of the at least one frame member in response to the stimulus once a stimulus threshold is reached. In one example, the one or more sensing elements comprises at least one of the following: at least one heart rate monitor, at least one blood pressure monitor, at least one light sensor, at least one photovoltaic element, at least one humidity sensor, and at least one chemical sensor.

In one aspect, the at least one heart rate monitor is configured to sense changes in heart rate to effect the color changing element.

In one aspect, the at least one blood pressure monitor is configured to sense changes in blood pressure to effect the color changing element.

In one aspect, the at least one photovoltaic element is configured to sense changes in light to effect the color changing element.

In one aspect, the at least one light sensor is configured to sense changes in light to effect the color changing element.

In one aspect, the at least one humidity sensor is configured to sense changes in humidity to effect the color changing element.

In one aspect, the at least one chemical sensor is configured to sense changes in chemical changes in an environment or in contact with the at least one frame member to effect the color changing element.

Other objects and advantages of the various embodiments of the present invention will become obvious and intuitive to the reader and it is intended that these objects and advantages are within the scope of the present invention.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a schematic of the dynamic glasses frame having a color changing element, wherein the dynamic glasses frame is changing from a first color to a second color, according to some embodiments; and

FIG. 2 is a block diagram of the sensing elements configured to effects the color changing element of the dynamic glasses frame, according to some embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing in detail exemplary embodiments, it is noted that the embodiments reside primarily in combinations of components related to particular devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In general, the embodiments provided here relate to a dynamic glasses frames and accessories which may include dynamic elements that allow for them to change colors and/or designs. These can reflect a user's style, individuality, biometrics, physiology, and/or the environment. The dynamic glasses frame may include at least one frame member comprising a color changing element to change from at least one first color to at least one second color in response to a stimulus. The at least one frame element may include a first temple, a second temple, and a frame, wherein the frame is coupled to one or more lenses.

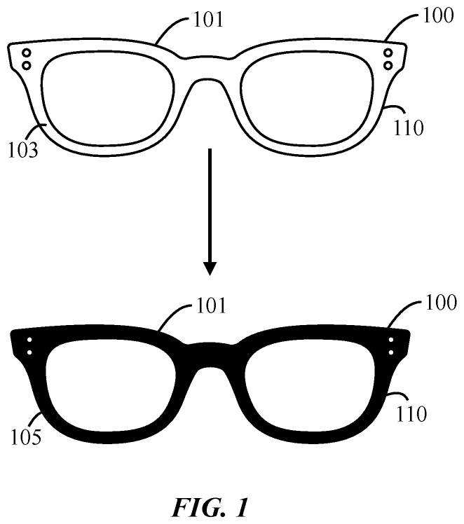

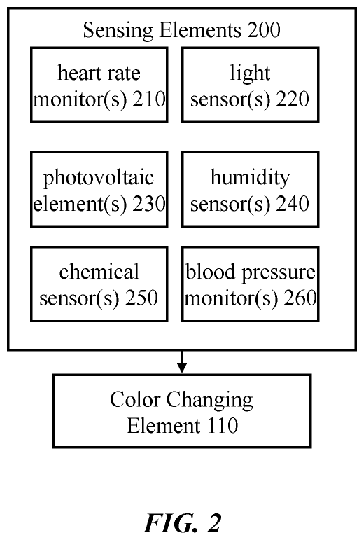

FIG. 1 illustrates a dynamic glasses frame 100 having at least one frame member 101 a color changing element 110 included therewith. The color changing element 110 may be at least partially embedded within the at least one frame member 101, removably coupled to the at least one frame member 101, or otherwise integrated into the at least one frame member 101. The at least one color changing element 110 may affect the color of the dynamic glasses frame 100 via changing the color of the dynamic glasses frame 100 (at least in part) from a first color 103 to a second color 105. For example, the first color 103 may be white while the second color 105 is black. The dynamic glasses frame may also include one or more sensing elements as shown and described in FIG. 2.

In some embodiments, the at least one frame member 101 may include a first temple and a second temple each pivotally coupled to a frame. The frame is coupled to at least one lens (often two lenses). One skilled in the arts will readily understand that the size, shape, and configuration of the at least one frame member 101 may take the form of nearly any common sunglass or eyeglass shape.

FIG. 2 illustrates a block diagram of the sensing elements 200 including a heart rate monitor(s) 210, light sensor(s) 220, photovoltaic elements 230, humidity sensor(s) 240, chemical sensor(s) 250, and blood pressure monitor(s) 260. The sensing elements 200 may receive the stimulus to change the color, via the color changing element 110, of the at least one frame member in response to the stimulus once a stimulus threshold is reached.

In some embodiments, the at least one light sensor 220 is configured to sense changes in light to effect the color changing element. For example, the at least one light sensor 220 may change the dynamic glasses frame from the first color (which is expressed during the day), to the second color (which is expressed during the night). Similarly, photovoltaic elements 230 may provide further electrical response to the stimulus.

In one aspect, the at least one heart rate monitor 210 is configured to sense changes in heart rate to effect the color changing element. For example, once the wearer has reached a stimulus threshold (in this embodiment defined as a heart rate in beats-per-minute) the dynamic glasses frame changes from the first color to the second color. In this example, this may aid in the user in determine if a target heart rate is reached during exercise.

In some embodiments, the at least one blood pressure monitor 260 is configured to sense changes in blood pressure to effect the color changing element. For example, once wearer has reached a blood pressure threshold (such as those which indicate health concerns) the color changing element changes the dynamic glasses frame from the first color to the second color.

In some embodiments, the at least one humidity sensor 240 is configured to sense changes in humidity to effect the color changing element. For example, once a stimulus threshold (corresponding to humidity) is reached, the color changing element changes the color of the dynamic glasses frame from the first color to the second color.

In some embodiments, the at least one chemical sensor 250 is configured to sense chemical changes in the environment or chemical contact with the dynamic glasses frame. For example, once a chemical is sensed, the color changing element changes the color of the dynamic glasses frame from the first color to the second color.

Glasses frames can be monolithic or otherwise be molded from and include a single member. Glasses frames can also be created using multiple members, including arms that may extend from a glasses face that holds the lens(es) in an area in front of the eyes and extend along the sides of the head and over the ears when worn by a user. These arms can be attached to or otherwise coupled with the glasses face using joints, hinges, and other mechanisms.

Glasses frames can be made from any number of natural and/or synthetic materials, including plastic, rubber, metal, wood, composites, and others. They may be created using injection molds, saws, drills, sanders, and any number of other tools, depending on their composition and requirements of the materials.

In many embodiments, glasses frames include dynamic elements that can comprise integrated thermochromic material(s) that change color due to ambient temperature in the environment and/or localized temperature due to proximity to the wearer's body, which may output different levels of heat in different environments. Other materials can also impact the level of heat that causes the color change, including hair, accessories such as hats, bandanas, bows, headbands, misters, cooling pads, and others.

In other embodiments, electronic components can be used to measure a wearer's heartrate, monitor ambient or localized sound, and others, and cause the dynamic elements to change color or design. Airflow such as wind can cause changes in some embodiments. Light changes can cause changes in some embodiments. Humidity or other exposure to liquid can cause changes in some embodiments. Electrical voltage and/or current can cause changes in some embodiments. Exposure to certain chemicals (in the air or directly) can cause changes in some embodiments.

In some embodiments portions of designs can be dynamic, while in others, whole designs themselves may be dynamic. For example, in an example embodiment a paisley design may change to a striped design in different temperatures. In other embodiments, loops in a paisley design may change color while the background remains the same. Different elements may be invisible in many environments but when heated or cooled, the elements may be revealed as the dynamic element materials react with the change in temperature.

Some dynamic elements may be finely tuned and reactive to change with minimal temperature or other changes, while other elements may be less susceptible to change and require greater change to exhibit their dynamic elements.

Also contemplated herein are accessories that can attach to one or more parts or portions of existing or later developed glasses frames and/or lenses. These can include plates, charms, or other accessories that can be permanently or removably coupled to the frames. In some embodiments these can be affixed with adhesives, clips, fasteners (e.g., hook and loop or others), connectors, magnets, and many others that are known or later developed. Shapes, styles, sizes, and other features can vary greatly in different embodiments. Some elements can hang off frames using chains, yarn, string, or many others to dangle below the frames.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety to the extent allowed by applicable law and regulations. The systems and methods described herein may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

The foregoing is provided for purposes of illustrating, explaining, and describing embodiments of this disclosure. Modifications and adaptations to these embodiments will be apparent to those skilled in the art and may be made without departing from the scope or spirit of this disclosure.

As used herein and in the appended claims, the singular forms “a”, “an”, and “the” include plural referents unless the context clearly dictates otherwise.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

In many instances entities are described herein as being coupled to other entities. It should be understood that the terms “coupled” and “connected” (or any of their forms) are used interchangeably herein and, in both cases, are generic to the direct coupling of two entities (without any non-negligible (e.g., parasitic) intervening entities) and the indirect coupling of two entities (with one or more non-negligible intervening entities). Where entities are shown as being directly coupled together or described as coupled together without description of any intervening entity, it should be understood that those entities can be indirectly coupled together as well unless the context clearly dictates otherwise.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described herein. A variety of modifications and variations are possible in light of the above teachings without departing from the following claims.

What is claimed is:

1. A dynamic glasses frame, comprising:
at least one frame member comprising a color changing element to change from at least one first color to at least one second color.

2. The dynamic glasses frame of claim 1, wherein the color changing element is integrated into the at least one frame member.

3. The dynamic glasses of claim 2, wherein the color changing element is at least partially embedded into the at least one frame member.

4. The dynamic glasses frame of claim 2, wherein the color changing element is removably coupled with the at least one frame member.

5. The dynamic glasses frame of claim 1, wherein the color changing element is in communication with at least one sensing element.

6. The dynamic glasses frame of claim 1, wherein the at least one frame element includes a first temple, a second temple, and a frame.

7. The dynamic glasses frame of claim 1, wherein the frame is coupled to one or more lenses.

8. A dynamic glasses frame, comprising:
at least one frame member comprising a color changing element to change from at least one first color to at least one second color in response to a stimulus,
wherein the at least one frame element includes a first temple, a second temple, and a frame, and wherein the frame is coupled to one or more lenses.

9. The dynamic glasses frame of claim 8, further comprising one or more sensing elements to receive the stimulus, the one or more sensing elements in communication with the color changing element to change the color of the at least one frame member in response to the stimulus once a stimulus threshold is reached.

10. The dynamic glasses frame of claim 9, wherein the one or more sensing elements comprises at least one of the following: at least one heart rate monitor, at least one blood pressure monitor, at least one light sensor, at least one photovoltaic element, at least one humidity sensor, and at least one chemical sensor.

11. The dynamic glasses frame of claim 10, wherein the at least one light sensor is configured to sense changes in light to effect the color changing element.

12. The dynamic glasses frame of claim 10, wherein the at least one heart rate monitor is configured to sense changes in heart rate to effect the color changing element.

13. The dynamic glasses frame of claim 10, wherein the at least one blood pressure monitor is configured to sense changes in blood pressure to effect the color changing element.

14. The dynamic glasses frame of claim 10, wherein the at least one photovoltaic element is configured to sense changes in light to effect the color changing element.

15. The dynamic glasses frame of claim 10, wherein the at least one light sensor is configured to sense changes in light to effect the color changing element.

16. The dynamic glasses frame of claim 10, wherein the at least one humidity sensor is configured to sense changes in humidity to effect the color changing element.

17. The dynamic glasses frame of claim 10, wherein the at least one chemical sensor is configured to sense changes in chemical changes in an environment or in contact with the at least one frame member to effect the color changing element.

18. The dynamic glasses frame of claim 8, wherein the color changing element is integrated into the at least one frame member.

19. The dynamic glasses of claim 8, wherein the color changing element is at least partially embedded into the at least one frame member.

20. The dynamic glasses frame of claim 8, wherein the color changing element is removably coupled with the at least one frame member.
